(19) United States
(12) Patent Application Publication
(10) Pub. No.: US 2019/0255309 A1
(43) Pub. Date: Aug. 22, 2019

(54) VALVE ASSEMBLY FOR BODY ACCESS DEVICE

(71) Applicant: pfm Medical, Inc., Carlsbad, CA (US)

(72) Inventor: David Stroup, Carlsbad, CA (US)

(21) Appl. No.: 16/280,929

(22) Filed: Feb. 20, 2019

Related U.S. Application Data
(60) Provisional application No. 62/633,387, filed on Feb. 21, 2018.

(51) Int. Cl. A61M 39/02; A61M 25/00

(52) U.S. Cl. CPC A61M 39/0247; A61M 25/0075; A61M 2039/0282; A61M 2039/027; A61M 25/0097

(57) ABSTRACT

A valved connector device can be attached with and/or incorporated into a body fluid access device, such as a fluid drainage sheath or catheter. The valved connector includes a valve that automatically opens when a secondary device, such as a catheter or tubing, is properly attached to the valved connector. When opened, the valved connector permits fluid flow therethrough so that fluid can be drained from a location, such as a fluid cavity of a patient. The valved connector is closed in a default state and automatically transitions to the closed state when the secondary device is decoupled from the valved connector. In this manner, hemostasis through the body fluid access device is achieved in the default state.

### CROSS REFERENCE TO RELATED APPLICATION

This application claims the benefit of U.S. Provisional Application Ser. No. 62/633,387 filed Feb. 21, 2018, the contents of which is incorporated herein by reference in its entirety and for all purposes.

### BACKGROUND

A volume of fluid can sometimes gather within a location in a patient's body. In such situations, a medical practitioner can use a catheter or other body cavity access device to drain such fluid from the patient's body. It can be desirable to limit, control, or otherwise regulate the flow of fluid through such catheters.

In this regard, a practitioner can use a valved connector which includes a fluid flow regulation valve to control the flow of fluids from the patient via the access device. The valved connector can fixedly or removably attach to the body access device.

There is a need for improvements in valved connectors to control the flow of fluid to and/or from a patient's body.

Disclosed herein is a valved connector device that can be attached with and/or incorporated into a body fluid access device, such as a fluid drainage sheath or catheter. The valved connector includes a valve that automatically opens when a secondary device, such as a catheter or tubing, is properly attached to the valved connector. When opened, the valved connector permits fluid flow therethrough so that fluid can be drained from a location, such as a fluid cavity of a patient. The valved connector is closed in a default state and automatically transitions to the closed state when the secondary device is decoupled from the valved connector. In this manner, hemostasis through the body fluid access device is achieved in the default state.

In one aspect, there is disclosed a valve assembly that controls the passage of fluid of a body access device, comprising: a body having an internal lumen with a proximal opening in the body and a distal opening in the body, wherein the internal lumen of the body is configured to be placed in fluid communication with a volume of fluid in a patient, wherein the body defines an internal chamber; a piston slidably positioned within the internal chamber; a first seal disk positioned in the internal chamber in a spaced relationship with the piston in a default state of the valve, the first disk including a slit that is closed in the default state, the slit sized to receive an elongated body of a secondary device therethrough; a separator disk juxtaposed with the first disk in the chamber, wherein the separator disk defines a space between the first disk and the separator disk; a second seal disk juxtaposed with separator disk such that the separator disk is interposed between the first seal disk and the second seal disk; and wherein the piston automatically moves toward and deforms the first seal disk so as to open the slit and permit passage of fluid therethrough so as to open the valve when the secondary device is coupled to the valve assembly.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIGS. 1 and 2 shows a catheter access system configured to allow fluid access to a body cavity from an extracorporeal location.

FIG. 3 shows the catheter access system in an exploded state.

FIG. 4 shows a perspective view of a valved connector of the system.

FIG. 5 shows a distal view of the valved connector.

FIG. 6 shows internal components as they are arranged within an outer housing of the valved connector.

FIG. 7 shows the internal components in an exploded state.

FIG. 8 shows the valved connector coupled to a sheath of the access system.

FIG. 9 shows a schematic representation of the collective assembly of the sheath and the valved connector inserted into a patient.

FIGS. 10 and 11 show cross-sectional view of the catheter access system in the region of the valve connector.

FIG. 12 shows a schematic representation of the collective assembly of the sheath, the valved connector, and the catheter inserted into a body cavity of the patient.

### DETAILED DESCRIPTION

Disclosed herein is a valved connector device that can be attached with and/or incorporated into a body fluid access device, such as a fluid drainage sheath or catheter. The valved connector includes a valve that automatically opens when a secondary device, such as a catheter or tubing, is properly attached to the valved connector. When opened, the valved connector permits fluid flow therethrough so that fluid can be drained from a location, such as a fluid cavity of a patient. The valved connector is closed in a default state and automatically transitions to the closed state when the secondary device is decoupled from the valved connector. In this manner, hemostasis through the body fluid access device in a default state.

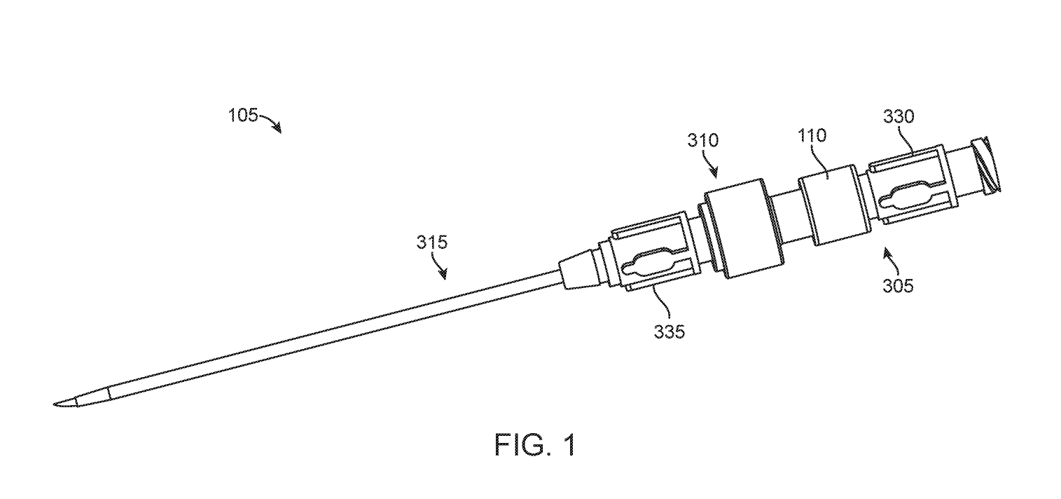

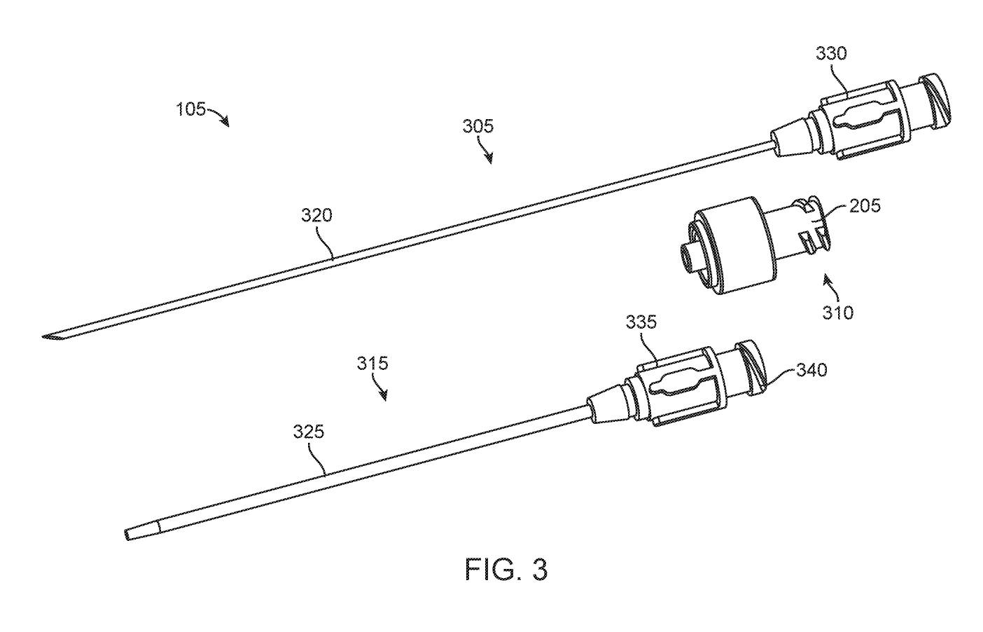

FIGS. 1-3 show a catheter body access system 105 configured to allow fluid access to a body cavity (such as a pleural space in the body of a human or animal patient) from an extracorporeal location. The access system 105 includes a valve assembly or valved connector 310 that provides a regulated, fluid flow interface for a lumen of a body access sheath 315 (FIG. 3) or any device that is configured to access fluid within a body. The body access sheath 315 is an elongated device with an internal lumen that can be placed in extracorporeal communication with a fluid-filled body cavity or other location of a patient. The internal lumen of the body access sheath 315 provides a passageway for fluid to be drained out of the body cavity. The valved connector 310, when coupled to the body access sheath 315, regulates flow of fluid into and out of the lumen of the body access sheath 315. A secondary access device, such as a catheter 305 (FIG. 3), can be coupled to the body access sheath 315, as described below.

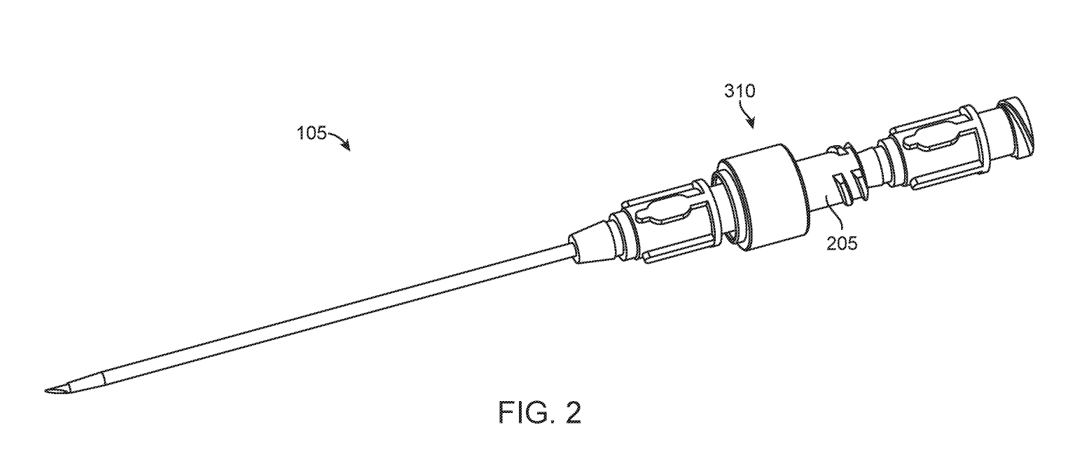

The valved connector 310, catheter 305, and sheath 315 are sized and shaped to be co-axially aligned and coupled to one another along a common long axis. FIG. 1 shows the valved connector 310, catheter 305, and sheath 315 in a coupled or assembled state so that the devices collectively form a unitary body. The devices include coupling mechanisms that permit a user to securely couple the devices to one another when co-axially aligned. For example, as shown in FIG. 1, the catheter 305 includes a coupler 110, such as a Luer-type connector, that removably connects and secures to a portion of the valved connector 310 in order to secure the catheter 305 to the valved connector 310 such as via a threaded interface. FIG. 2 shows the system 105 with the coupler 110 removed from the catheter 305 in order to show a threaded interface 205 of the valved connector 310, wherein the threaded interface 205 attaches to the coupler 110 of the catheter 305.

FIG. 3 shows the access system 105 in an exploded state with the valved connector 310, catheter 305, and sheath 315 separated from one another. The catheter 305 includes an elongated body, such as a needle 320, that has a hub 330 on a proximal end. As mentioned, the hub 330 has a coupler 110 (shown in FIG. 1) that can removably couple to a complementary threaded interface 205 of the valved connector 310. In the illustrated, example embodiment, the coupler 110 couples to the threaded interface 205 in a male-female relationship. The coupler 110 is rotatably attached to the hub 330 so that can rotate and threadedly attach to the threaded interface 205 of the valved connector 310.

With reference still to FIG. 3, the needle 320 of the catheter 305 has an internal lumen that runs the length of the needle 320 and that communicates with the hub 330. Fluid can flow into and out of the internal lumen of the needle 320 via a distal opening in the needle such as at a distal-most tip of the needle 320. An internal lumen of the hub 330 communicates with the internal lumen of the needle 320 and also communicates with a distal opening of the hub 330. In this manner, fluid can flow into the internal lumen of the needle and into the hub 330 and eventually out of the distal opening of the hub 330. The system 105 can further include a plug that inserts into the distal opening of the hub 330 to seal the opening closed.

As shown in FIG. 3, the sheath 315 includes an elongated cannula 325 with a cannula hub 335 on its proximal end. The cannula hub 335 has a threaded interface 340 that couples to a distal portion of the valved connector 310 such as in a threaded, male-female relationship. The cannula 325 of the sheath 315 has an internal lumen that runs the length of the cannula 325 and that communicates with the cannula hub 335. Fluid can flow into and out of the internal lumen of the cannula 325 via a distal opening in the cannula 325 such as at a distal-most tip of the cannula 325. An internal lumen of the hub 335 communicates with the internal lumen of the cannula 325 and also communicates with a distal opening of the hub 335. When the valved connector 310 is attached to the sheath 315, the valved connector 310 controls fluid flow through the internal lumen of the sheath 315, as described below.

The internal lumen of the cannula 315 is sized and shaped to co-axially receive the needle 320 of the catheter 305. In this manner, the sheath 315 and catheter 305 can be co-axially aligned when the needle 320 is inserted into the cannula 325. The needle 320 can have a length that is longer than the length of the cannula 325 so that a distal end of the needle 320 pokes out of the distal end of the cannula in the assembled device, such as shown in FIG. 1.

As shown in FIG. 1, the valved connector 310 can be positioned between the hub 335 of the sheath 315 and the hub 330 of the catheter 305. The valved connector lockingly secures to the hub 335 of the sheath 315 and the hub 330 of the catheter 305. When aligned and secured as such, the needle 320 of the catheter is positioned co-axially within the cannula 325 of the sheath with the valved connector 310 acting as a valved interface between the sheath 315 and the catheter 305.

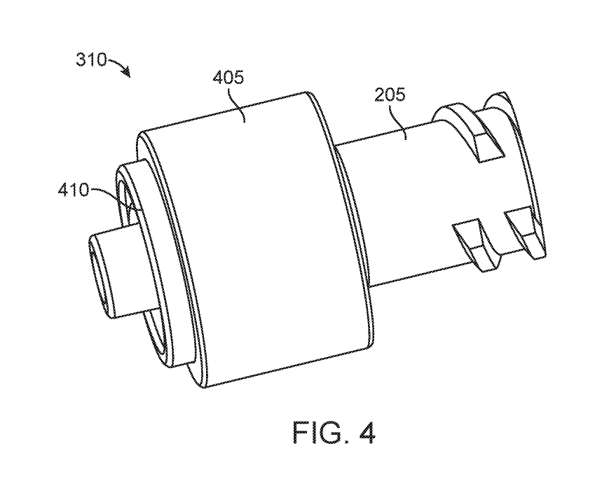

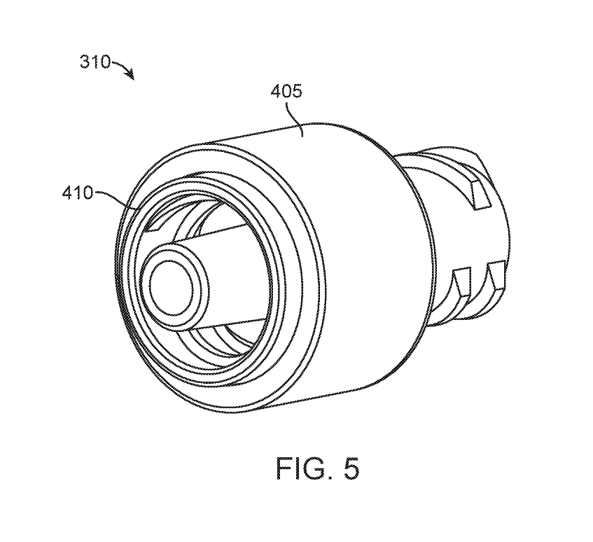

FIG. 4 shows a perspective view of the valved connector 310. FIG. 5 shows another perspective view of the valved connector 310. The valved connector 310 includes an outer housing 405 having a main body from which a neck extends. In the illustrated embodiment, the neck is circular in cross-section and has a smaller diameter than the diameter of a distal portion of the main body 405. The neck forms the threaded interface 205, which securely and sealingly couples to the coupler 110 (FIG. 1) of the catheter 305 in a male-female relationship such as by rotating relative to one another. A distal interface 410 of the valved connector 310 is configured to couple to the threaded interface 340 (FIG. 3) of the cannula hub 335, as described further below. In the illustrated embodiment, the outer housing is substantially cylindrical although the shape may vary.

As shown in the proximal view of FIG. 5, the distal interface 410 of the valved connector has a threaded region (such as a threaded female surface), which is configured to couple in a rotatable, threaded manner to the threaded interface 340 (FIG. 3) of the cannula hub 335. That is, the threaded interface 340 inserts into the distal interface 410 of the valved connector and the two securely and sealingly couple to one another by a threaded engagement. When coupled as such, the internal lumen of the cannula 315 fluid communicates with an internal lumen of valved connector 310 such that fluid must flow through the valved connector in order to flow into or out of the internal lumen of the cannula 315 via the cannula hub 340.

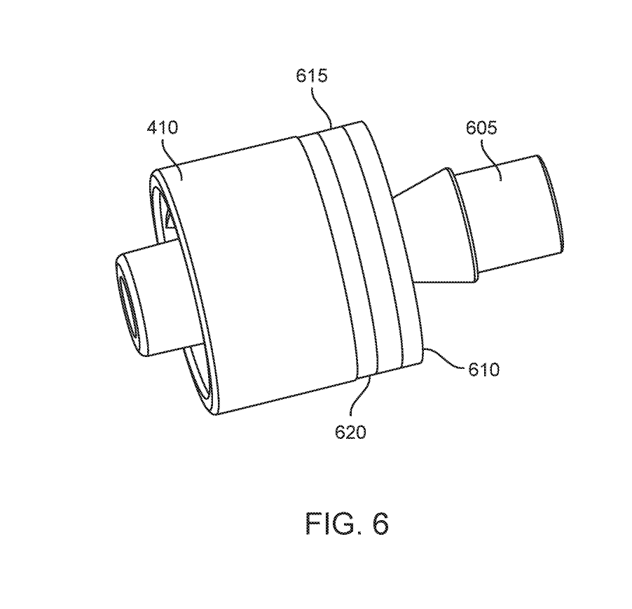

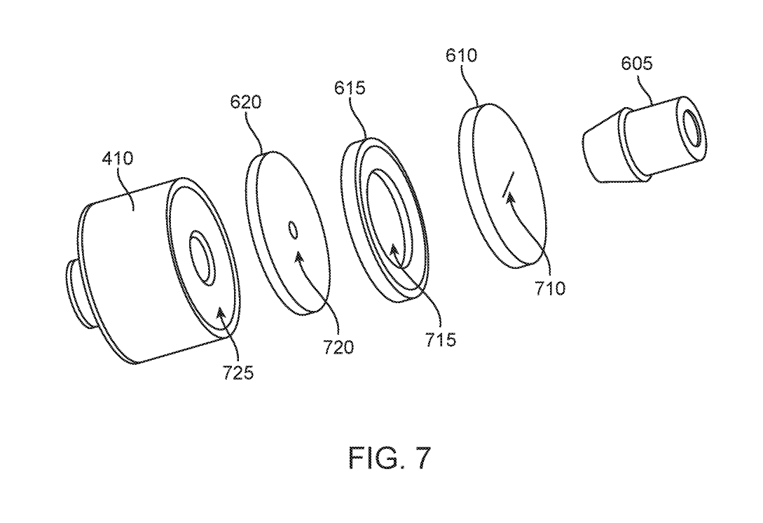

The outer housing 405 of the valved connector 310 defines an internal lumen that contains several components of the valved connector 310, wherein the components control fluid flow through the valved connector 310. FIG. 6 shows the internal components as they are arranged within an internal chamber of the outer housing 405 of the valved connector 310. The outer housing of the valved connector 310 is not shown in FIG. 6 for clarity of illustration. FIG. 7 shows the internal components in an exploded state.

With reference to FIGS. 6 and 7, the internal components include an elongated piston 605 having an internal lumen, which co-axially aligns with the long axis of the assembled system. The piston 605 has a substantially cylindrical shape with a sloped, distal end that reduces in transverse dimension (relative to the long axis) moving in a distal direction. That is, a diameter of the piston gradually reduces or tapers moving in a distal direction. In the assembled valved connector 310, the piston 605 is slidably positioned within the neck of the outer housing 405.

The internal components further include a round first seal disk 610 made of a malleable material that can seal with the piston 605 when in contact therewith. The first seal disk 610 has a slit 710 that extends through the seal disk 610. The seal disk is made of a material such that the slit seals shut in a default state and can also be deformed and/or forced open such as when contacted with sufficient force by the distal end of the piston. The slit 710 is sized to receive therethrough the needle 320 of the catheter 305.

The internal components further include a separator disk 615 positioned in a juxtaposed, contacting relationship with the first seal disk 610 and a second seal disk 620. The separator disk 615 is interposed between the first seal disk 610 and second seal disk 620 in the assembled state, as shown in FIG. 6. The separator disk 615 has a central aperture 715 with a chamfered, annular surface that surrounds the aperture 715 on both sides of the separator disk. The chamfered surface of the separator disk 615 is such that a space is formed in the region of the chamfered surface between both the second seal disk 620 and the separator disk 615, and also the first seal disk 610 and the separator disk 615. The chamfered surface can exist on one or both sides of the separator disk 615.

The second seal disk 620 also has a central aperture 720 that is sized and shaped to snugly receive therethrough the needle 320 of the catheter 305. The second seal disk 620 is also made of a resilient, flexible material.

With reference still to FIGS. 6 and 7, the distal interface 410 of the valved connector 310 has a front face 725 that is juxtaposed with the second seal disk 620 (in the assembled state) such that a central opening in the distal interface 410 co-axially aligns with the central apertures 720 and 715 and also with the slit 710. The distal interface 410, second seal disk 620, separator disk 615, and first seal disk 610 all have round outer shapes that fit snug within the main body 405 (FIGS. 4 and 5) in the assembled state. The piston 605 is slidably positioned within the neck of the main body 405 in the assembled state.

Although FIGS. 1-3 show the access system 105 including a secondary access device comprising a catheter 305, it should be appreciated that variety of types and configurations of secondary access devices can be utilized in connection with valved connector 310. For example, the valved connector 310 can be attached to an access device that does not having a threaded connector. The valved connector 310 can also be secured to a catheter, tubing, or other apparatus or can be secured to an apparatus that is not directly in fluid communication with the volume of fluid in a body, but that is instead connected to a secondary apparatus. The valved connector can also be used to control the flow of fluid from one location to another location. For example, the valved connector can be used with an infusate bag or medical tubing that is not in communication with a patient's body. In another embodiment, the valved connector 310 can be an integral part of the sheath 315 such that the valved connector is not removable from the sheath 315. The valved connector 310 can also be attached upstream or downstream of the valved connector via tubing.

In use, the valved connector 310 is coupled to the sheath 315 such as by attaching the distal interface 410 of the valved connector 310 to the threaded interface 340 at the proximal end of the sheath 315. As mentioned, the distal interface 410 can be rotatably attached in a threaded manner to the threaded interface 340 to securely attach the valved connector 310 to the sheath 315. As mentioned, when attached as such, the internal lumen of the cannula 325 co-axially aligns with the internal lumen of the valved connector 310. In this manner, the valved connector 310 controls fluid flow (such as by inhibiting, permitting, or blocking flow) through the sheath 315 and valved connector based upon the state of the internal components of the valved connector. In an alternate embodiment, the valve connector 310 is integrally formed as part of the sheath 315.

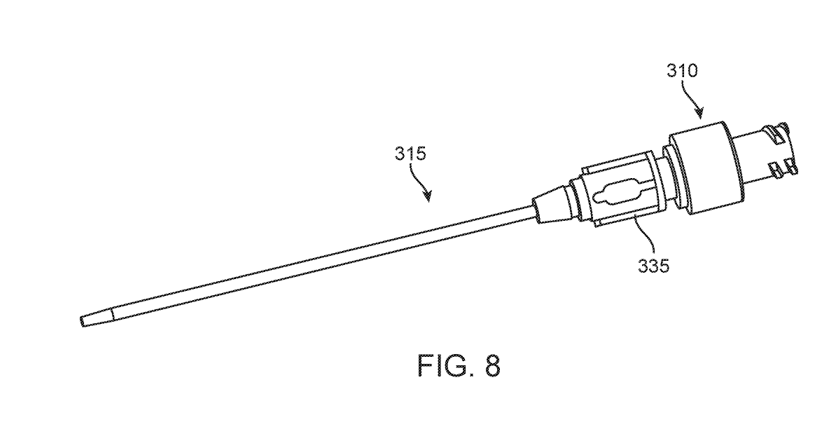

FIG. 8 shows the sheath 315 and the valved connector 310 attached to one another after the valved connector 310 has been coupled to the hub 335 of the sheath 315. As mentioned, the valved connector 310 attaches to the proximal end of the sheath 315 at the cannula hub 335. Once attached as such, the collective assembly of the sheath 315 and the valved connector 310 can be inserted by a user into fluid communication with a body cavity of a patient.

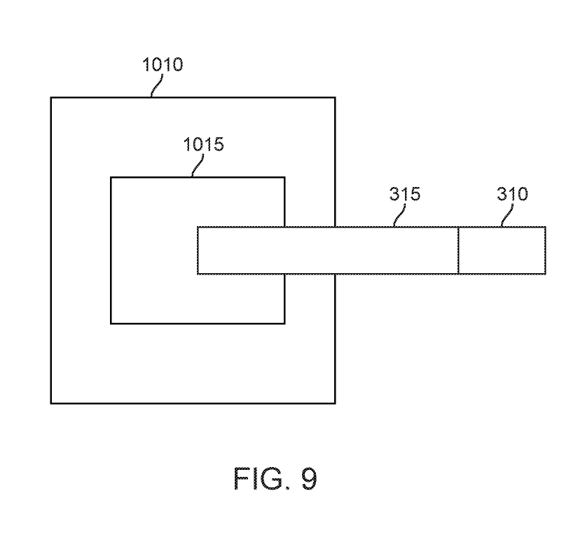

FIG. 9 shows a schematic representation of the collective assembly of the sheath 315 and the valved connector 310 inserted into a patient 101 such that the sheath 315 communicates with a body cavity 1015 of the patient 1010, wherein the body cavity 1015 is filled at least partially with fluid. The body cavity 1015 can be any fluid-filled cavity within the patient. In an embodiment, the body cavity 1015 is a pleural space of a patient. The distal end of the cannula of the sheath 315 is positioned within the body cavity 1015 such that the internal lumen of the sheath 315 fluidly communicates with the body cavity 1015. In this manner, the sheath 315 provides extracorporeal access to the body cavity 1015. Fluid of the body cavity 1015 can flow into the internal lumen of the sheath 315 wherein the valved connector 310 controls or regulates fluid flow out of the sheath 315. As mentioned, in a default state, the valved connector 310 is closed such that it blocks fluid from flowing out of the sheath 315. A lumen of the valved connector 310 is collectively formed by the central apertures 720 and 715 (FIG. 7) and the slit 710. This internal lumen of the valved connector 310 is initially closed as a result of bulk resilience of the material of the first disk 610 maintaining the slit 710 in a sealed, close state.

The valved connector 310 can be opened for fluid flow therethrough by coupling a secondary device, such as the catheter 305, to the valved connector 310. As the secondary device, such as the catheter 305, is inserted into the valved connector 310, a portion of the secondary device directly or indirectly deforms the slit 710 to thereby open the slit 710 and allow fluid flow therethrough. The process of coupling secondary device to the valved connector 310 and sheath 315 is now described in the example context of coupling the catheter 305 to the valved connector 310 and sheath 315.

The catheter 305 is coupled to the valved connector 310 by inserting the needle 320 of the catheter 305 into the internal lumen of the valved connector 310 so that the sheath 315, valved connector 310, and catheter 305 collectively form a single assembly, as shown in FIG. 1. As mentioned, the various coupling components can be secured to one another to thereby secure the devices in an assembled state. The catheter 305 can be advance distally into and relative to the valved connector 310 and the sheath 315 such that the needle 320 advances into the internal lumen of the valved connector 310 and the internal lumen of the sheath 315. As the catheter 305 is coupled or advanced into to the valve connector 310, the catheter automatically transitions the valved connector 310 to the open state so that fluid can flow therethrough.

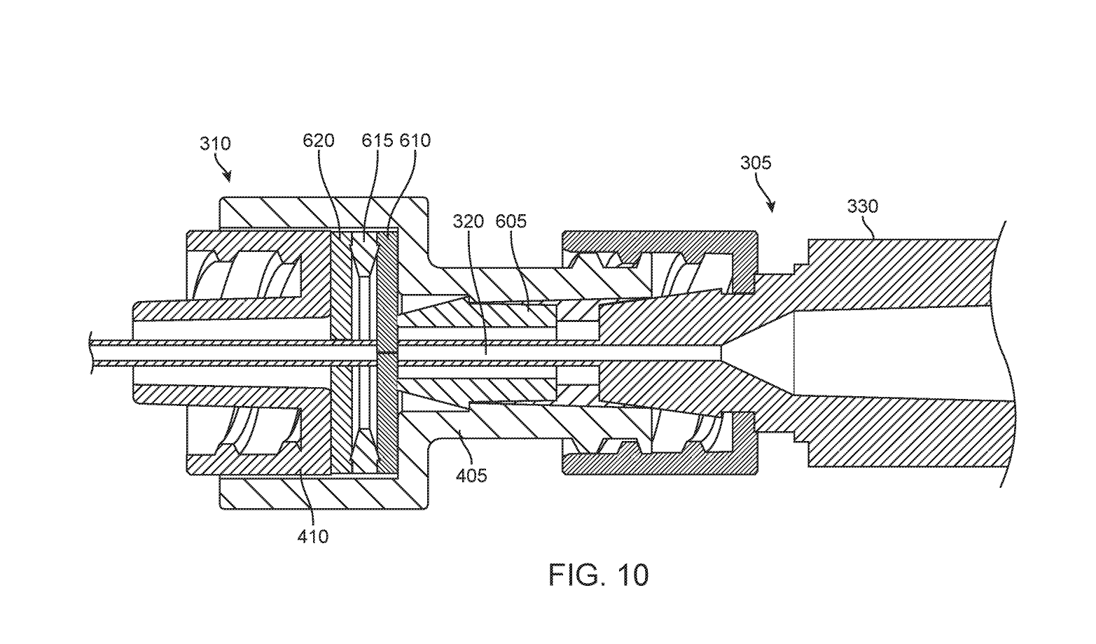

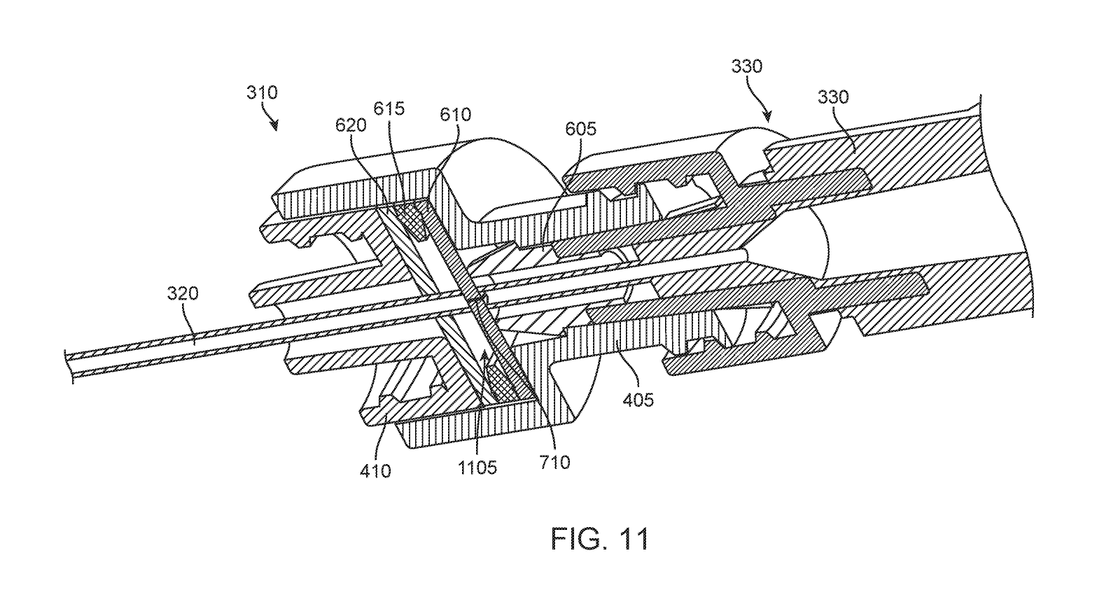

This is described in more detail with reference to FIGS. 10 and 11, which show cross-sectional view of the catheter access system 105 in the region of the valve connector 310 with the needle 320 of the catheter 305 inserted through the valved connector 310. The cannula 315 is not shown in FIGS. 10 and 11 for clarity of illustration. As the catheter 305 advances distally, a portion of the catheter 305 (such as a distal portion of the hub 330) abuts and pushes the piston 605 in a distal direction toward the first seal disk 610.

The distal end of the piston 605 moves distally (through the neck of the main body 405) so that it contacts the first seal disk 610. The distal end of the piston 605 exerts a force om the first seal disk 610 so as to deform the first seal disk 610 in a manner that opens the slit 710 (FIG. 11). As mentioned, the separator disk 615 is shaped so that a space 1105 (FIG. 11) is formed between the first seal disk 610 and the separator disk 615. This space provides for a region of movement and deformation of the first seal disk 610 as the piston 605 pushes against the first seal disk 506. The space 1105 is sufficiently large such that the first seal disk 610 can sufficiently deform within the space 1105 so that the slit 710 opens and provides a passageway for fluid to flow therethrough.

This creates a fluid passageway within the valved connector 310 through the slit 710 in a region that surrounds the needle 320 with the fluid passageway collectively formed by formed by the central apertures 720 and 715 (FIG. 7) and the slit 710 within the valved connector 310. Fluid can pass through this fluid passageway, which automatically opens upon coupling of the catheter 305 (or other secondary device) to the valved connector 310. Upon removal or de-coupling of the catheter 305 from the valved connector 310, the piston 605 automatically disengages from contact with the first seal disk 605. As mentioned, the first seal disk is made of a material such that the slit will automatically return to a closed state when the first seal disk is not deformed. In this manner, the first seal disk 610 and the valved connector 310 automatically close to prevent fluid flow therethrough upon removal of the catheter 305.

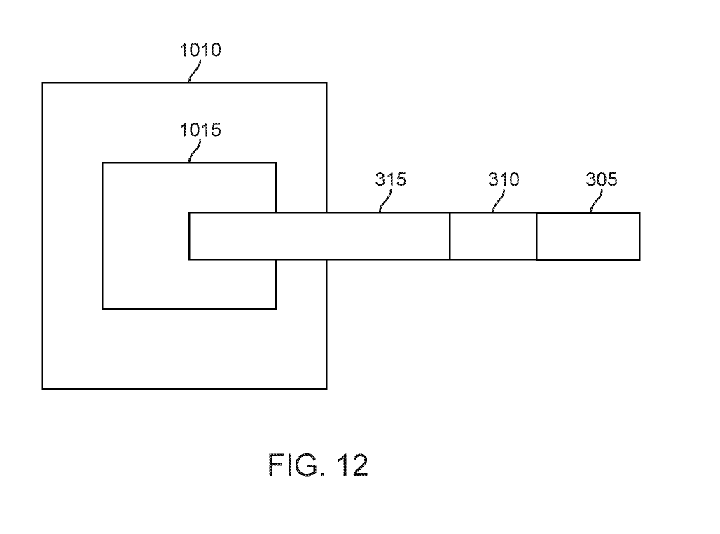

FIG. 12 shows a schematic representation of the collective assembly of the sheath 315, the valved connector 310, and the catheter 305 (or other such secondary access device) inserted into a body cavity 1015 of the patient 1010. As mentioned, the sheath 315 communicates with the body cavity 1015 such that the internal lumen of the sheath 315 can be used to drain fluid out of the body cavity 105. With the catheter 305 coupled to the valve connector 310, the valved connector 310 is automatically opened to permit fluid flow out of the sheath 315. As mentioned, other secondary devices aside from the catheter 305 can be coupled to the valved connector 310. The catheter 305 is just a non-limiting example.

A variety of types of mechanisms can be used with the devices herein. For example, a connector that is not threaded can be secured to the distal end of the valved connector. Both the proximal and distal ends of the valved connector can have the same type of securement configuration. For example, both ends of the valved connector can have a female connector or both ends can have a male connector. In another embodiment, both ends can include a compression or other fitting that does not require threads. Other known connection or securement type configurations can be used.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A valve assembly that controls the passage of fluid of a body access device, comprising:
a body having an internal lumen with a proximal opening in the body and a distal opening in the body, wherein the internal lumen of the body is configured to be placed in fluid communication with a volume of fluid in a patient, wherein the body defines an internal chamber;
a piston slidably positioned within the internal chamber;
a first seal disk positioned in the internal chamber in a spaced relationship with the piston in a default state of the valve assembly, the first disk including a slit that is closed in the default state, the slit sized to receive an elongated body of a secondary device therethrough;
a separator disk juxtaposed with the first disk in the chamber, wherein the separator disk defines a space between the first disk and the separator disk;
a second seal disk juxtaposed with separator disk such that the separator disk is interposed between the first seal disk and the second seal disk;
wherein the piston automatically moves toward and deforms the first seal disk so as to open the slit and permit passage of fluid therethrough such that the valve assembly automatically opens when the secondary device is coupled to the valve assembly.

2. The valve assembly of claim 1, wherein the piston automatically moves back to the default state upon uncoupling of the secondary device is coupled from the valve assembly.

3. The valve assembly of claim 1, wherein at least a portion of the secondary device pushes the piston toward the first seal disk as the secondary device is coupled to the valve assembly.

4. The valve assembly of claim 1, wherein the secondary device is a catheter having a needle, and wherein the needle inserts though the valve assembly when the catheter is coupled to the valve assembly.

5. The valve assembly of claim 1, wherein the first seal disk is made of plastic.

6. The valve assembly of claim 1, wherein the body is at least partially cylindrical.

7. The valve assembly of claim 6, wherein the body has a neck portion in which the piston is slidably positioned.

8. The valve assembly of claim 1, wherein the body is placed in fluid communication with the volume of fluid in the patient via a body access device that is placed in fluid communication with the volume of fluid.

9. The valve assembly of claim 8, wherein the body access device is a body access sheath having an internal lumen that couples to the internal lumen of the body.

10. The valve assembly of claim 8, wherein the body access device removably attaches to the secondary device.

11. A method of controlling the passage of fluid of a body access device, comprising:
providing a valve assembly having:
a body having an internal lumen with a proximal opening in the body and a distal opening in the body, wherein the internal lumen of the body is configured to be placed in fluid communication with a volume of fluid in a patient, wherein the body defines an internal chamber;
a piston slidably positioned within the internal chamber;
a first seal disk positioned in the internal chamber in a spaced relationship with the piston in a default state of the valve, the first disk including a slit that is closed in the default state, the slit sized to receive an elongated body of a secondary device therethrough;
a separator disk juxtaposed with the first disk in the chamber, wherein the separator disk defines a space between the first disk and the separator disk;
a second seal disk juxtaposed with separator disk such that the separator disk is interposed between the first seal disk and the second seal disk;
attaching a body access device to the valve assembly;
coupling the secondary device to the valve assembly by inserting the secondary device into the internal lumen of the body of the valve assembly such that insertion of the secondary device causes the piston of the valve assembly to move toward and deform the first seal disk of the valve assembly so as to open the slit and permit passage of fluid therethrough so as to open the valve assembly when the secondary device is coupled to the valve assembly.

12. The method of claim 11, wherein the body access device is a fluid drainage sheath.

13. The method of claim 11, wherein the body access device is a catheter.

14. The method of claim 11, wherein the secondary device is a catheter.
